(19) United States
(12) Patent Application Publication
(10) Pub. No.: US 2017/0143017 A1
(43) Pub. Date: May 25, 2017

(54) BEADLETS COMPRISING CAROTENOIDS

(71) Applicant: DSM IP ASSETS B.V., Heerlen (NL)

(72) Inventors: Gabriela BADOLATO BOENISCH, Basel (CH); Bernd SCHLEGEL, Basel (CH)

(21) Appl. No.: 15/400,689

(22) Filed: Jan. 6, 2017

Related U.S. Application Data
(62) Division of application No. 14/436,249, filed on Apr. 16, 2015.

(30) Foreign Application Priority Data
Oct. 18, 2012 (EP) 12189045.3

(51) Int. Cl. A23L 5/44; A23D 7/005; A23L 2/58; A61K 8/92; A61K 8/02; A61Q 19/00; A61K 8/31; B01J 2/04; A23K 20/179

(52) U.S. Cl. CPC A23L 5/44; B01J 2/04; A23D 7/0056; A23L 2/58; A23K 20/179; A61K 8/0241; A61Q 19/00; A61K 8/31; A61K 8/922; A23V 2002/00; A61K 2800/522; A61K 2800/43

(57) ABSTRACT

The present invention relates to process for producing beadlets comprising at least one carotenoid and matrix material, which comprise wax(es) and/or fat(s) with a melting point of between 40 to 85° C., as well as to the production of such beadlets and to the use of such beadlets in compositions.

This application is a divisional of commonly owned copending U.S. Ser. No. 14/436,249, filed Apr. 16, 2015, which is the national phase application of international application PCT/EP2013/071809, filed Oct. 18, 2013, which designated the US and claims priority to European Patent Application 12189045.3, filed Oct. 18, 2012, the entire contents of each of which are hereby incorporated by reference.

The present invention relates to beadlets comprising at least one carotenoid and matrix material, which comprise wax(es) and/or fat(s) with a melting point of between 40 to 85° C., as well as to the production of such beadlets and to the use of such beadlets in compositions. Nowadays there are many forms of formulations available, which comprise carotenoids. Solid, liquid or paste-like formulations are known. These formulations do have disadvantages. The liquid formulations do have a tendency to get inhomogenously, so that they have to be shaken regularly to avoid that.

The problems with the solid formulations are for example
i) dust issues (not easy to handle and risk of explosion),
ii) the particles stick together.

The goal of the present invention was to find a form of formulation, which is easy to produce and which is also good and safe to handle.

It was found that when a formulation in the form of beadlets, which comprise at least one carotenoid and a matrix (waxes and/or fats with specific and well defined properties), was produced then a formulation was obtained which does not have the disadvantages as mentioned above.

And furthermore, such beadlets can be produced by using the spray chilled process, which is a mild production procedure. The principle of this process is widely known.

The main advantages of the beadlets according to the present invention are the following
(i) good and fast dispersibilty into an oil (phase); and
(ii) good flowabilty (not sticky, not dusty and easy to dose); this property result in essentially no material loss when transferred from one container to another and a container, wherein the beadlets have been stored can be cleaned easily; and
(iii) the color saturation as well as the color stability of the beadlets in an end-market product (consumer product, which is sold in (grocery) shops) is good (also after thermal treatment, such for example pasteurization).

Therefore, the present invention relates to beadlets (I) which comprise
(i) 25 weight-% (wt-%) to 45 wt-%, based on the total weight of the beadlets, of at least one carotenoid, and
(ii) 55 wt-% to 75 wt-%, based on the total weight of the beadlets, of a matrix material comprising at least one wax and/or fat, wherein the wax and/or fat are unsaturated (non-hydrogenated), partially saturated (partially hydrogenated) or fully saturated (fully hydrogenated),
   characterized in that the matrix material has a melting point of from 40 to 85° C.

It is clear that the percentages always add up to 100%. This requirement applies to all compositions.

Further preferred are beadlets (Ia), which are beadlets (I),

with the proviso that beadlets comprising
(i) 33 wt-% to 34 wt-%, based on the total weight of the beadlets, of β-carotene, and
(ii) 66 wt-% to 67 wt-%, based on the total weight of the beadlets, of glycerin monostearate,
   are excluded from the scope of the claims.

Beadlets (comprising fat-soluble substances) and their methods of productions are known from the prior art, for example from US 2006/0115534 and U.S. Pat. No. 4,670,247. These beadlets usually have good storage stability, but the concentration of the fat-soluble substances in such beadlets is low. Usually the content is between 5 to 15 wt-%, based on the total weight of the beadlets.

With the present invention it was possible to form non-sticky, non dusty beadlets by using the cold spray drying granulation technology. Furthermore, these beadlets can comprise up to 45 wt-%, based on the total weight of the beadlets, of at least one carotenoid.

Preferred amounts of at least one carotenoid in the beadlets according to the present invention are 25 wt-%-40 wt-% and 25 wt-%-35 wt-% (all based on the total weight of the beadlets).

As a consequence thereof the preferred amounts of matrix material comprising at least one wax and/or fat, wherein the wax and/or fat are unsaturated (non-hydrogenated), partially saturated (partially hydrogenated) or fully saturated (fully hydrogenated), characterized in that the matrix material has a melting point of from 40 to 85° C., are 60 wt-%-75 wt-% and 65 wt-%-75 wt-%, (all based on the total weight of the beadlets).

Therefore preferred embodiments of the present invention are beadlets (II) which comprise
(i) 25 wt-%-40 wt-%, based on the total weight of the beadlets, of at least one carotenoid, and
(ii) 60 to 75 wt-%, based on the total weight of the beadlet,s of a matrix material comprising at least one comprising at least one wax and/or fat, wherein the wax and/or fat are unsaturated (non-hydrogenated), partially saturated (partially hydrogenated) or fully saturated (fully hydrogenated), characterized in that the matrix material has a melting point of from 40 to 8520 C.

Further preferred are beadlets (III), which are beadlets (II),

with the proviso that beadlets comprising
(i) 33 wt-% to 34 wt-%, based on the total weight of the beadlets, of β-carotene, and
(ii) 66 wt-% to 67 wt-%, based on the total weight of the beadlets, of glycerin monostearate,
   are excluded from the scope of the claims.

Therefore also preferred embodiments of the present invention are beadlets (III) which comprise
(i) 25 wt-%-35 wt-%, based on the total weight of the beadlets, of at least one carotenoid, and
(ii) 65 wt-%-75 wt-%, based on the total weight of the beadlets of a matrix material comprising at least one comprising at least one wax and/or fat, wherein the wax and/or fat are unsaturated (non-hydrogenated), partially saturated (partially hydrogenated) or fully saturated (fully hydrogenated), characterized in that the matrix material has a melting point of from 40 to 85° C.

Further preferred are beadlets (IIIa), which are beadlets (III), with the proviso that beadlets comprising
(i) 33 wt-% to 34 wt-%, based on the total weight of the beadlets, of β-carotene, and
(ii) 66 wt-% to 67 wt-%, based on the total weight of the beadlets, of glycerin monostearate,
   are excluded from the scope of the claims.

The term “beadlet(s)” as used herein refers to small discrete particles, which have a mean (average) particle size of 50 μm to 1000 μm in diameter (preferably from 250 μm to 850 μm). The sizes can be smaller or larger. The size of a beadlet can be determined according to well known methods, such as (scanning) electron microscopy or laser diffraction.

All beadlets sizes (=particle size distribution (PSD)) in the present patent application have been determined by laser diffraction”. The PSD was done with analyzer Malvern Mastersizer 2000 and sampler Scirocco 2000. The testing method involve calculation model Fraunhofer with settings “General Purpose”, “Normal Sensitivity”, “No Air Pressure”, “Obscuration 5-10%”. Beadlets are usually nearly spherical. Beadlets contain one or more active ingredients in an encapsulated form.

Therefore a further embodiment of the present invention relates to beadlets (IV), which are beadlets (I), (Ia), (II), (IIa), (III) and/or (IIIa), which have mean (average) particle sizes of 50 μm to 1000 μm in diameter (preferably from 250 μm to 850 μm).

The beadlets according to the present invention comprise at least one carotenoid. The term “carotenoid” as used herein comprises a carotene or structurally related polyene compound which can be used as a colorant for food, beverages, animal feeds, cosmetics or drugs. Examples of such carotenoids are α- or β-carotene, 8′-apo-β-carotenal, 8′-apo-β-carotenoic acid esters such as the ethyl ester, canthaxanthin, astaxanthin, lycopene, lutein, zeaxanthin or crocetin, or mixtures thereof. The preferred carotenoid is β-carotene.

Therefore a preferred embodiment of the present invention relates to beadlets (V), which are beadlets (I), (Ia), (II), (IIa), (III), (IIIa) and/or (IV), wherein the at least one carotenoid is chosen from the group consisting of α- or β-carotene, 8′-apo-β-carotenal, 8′-apo-β-carotenoic acid esters (such as the ethyl ester), canthaxanthin, astaxanthin, lycopene, lutein, zeaxanthin and crocetin.

A more preferred embodiment of the present invention relates to beadlets (IV), which are beadlets (I), (Ia), (II), (IIa), (III), (IIIa), (IV), (V) and/or (VI), wherein the carotenoid is β-carotene.

It is clear that the percentages always add up to 100%.

The beadlets according to the present invention comprise at least one unsaturated (non-hyrdogenated), partially saturated (partially hydrogenated) or fully saturated (fully hydrogenated) wax and/or unsaturated (non-hydrogenated), partially saturated (partially hydrogenated) or fully saturated (fully hydrogenated) fat with a melting point of from 40 to 85° C., preferably 45 to 80° C.

Waxes in the context of the present invention are organic compounds that characteristically consist of a long alkyl chains. Natural waxes (plant, animal) are typically esters of fatty acids and long chain alcohols. Synthetic waxes are long-chain hydrocarbons lacking functional groups.

Fats consist of a wide group of compounds that are generally soluble in organic solvents and largely insoluble in water. Hydrogenated (or saturated fats) in the context of the present invention are generally triesters of glycerol and fatty acids. Fatty acids are chains of carbon and hydrogen atoms, with a carboxylic acid group at one end. Such fats can have natural or synthetic origin. It is possible to hydrogenate a (poly)unsaturated fat to obtain a hydrogenated (saturated) fat.

The matrix which comprises at least one wax and/or fat has a melting point of 40 to 85° C. (preferably 45 to 80° C.). The melting point of a wax/fat in the context of the present invention is usually not a sharp point. It is more a range, due to the fact that most fats/waxes are a mixture of different chain lengths. The determination of the melting point is carried out as described in the standard norm ISO 6321:2002.

Preferred examples of unsaturated (non-hydrogenated), partially saturated (partially hydrogenated) or fully saturated (fully hydrogenated) fats and waxes suitable for the present invention are glycerin monostearate, carnauba wax, candelilla wax, palmitic acid, stearic acid (i.e. Parteck LUB STA 50® from Merck Milipore), glycerintristearate, glycerin mono-, di-, tribehenate, behenic acid, polyclyceryl palmito stearate, Revel A® (hydrogenated, refined vegetable fat, made out of palm oil from Loders Croklaan), Revel C® (fractionated, not hydrogenated, refined vegetable fat, made out of palm oil; from Loders Croklaan) and hydrogenated rapeseed oil (hydrogenated colza oil). These compounds can be used as such or as mixtures. Especially preferred are glycerin monostearate, Revel A®, Revel C®, hydrogenated rapeseed oil, palmitic acid.

Most of these listed matrix materials are commercially available.

Therefore a preferred embodiment of the present invention relates to beadlets (XIII), which are beadlets ((I), (Ia), (II), (IIa), (III), (IIIa), (IV), (V), (VI), (VII), (VIII), (IX), (X), (XI), (XII) and/or (XII′), wherein the at least one wax and/or fat having a melting point of 40 to 85° C. (preferably 45 to 80° C.), is chosen from the group consisting of glycerin monostearate, candelilla wax, palmitic acid, stearic acid, glycerintristearate, glycerin mono-, di-, tribehenate, behenic acid, polyclyceryl palmito stearate, Revel A® (hydrogenated, refined vegetable fat, made out of palm oil), Revel C® (fractionated, not hydrogenated, refined vegetable fat, made out of palm oil) and hydrogenated rapeseed oil (hydrogenated colza oil).

The beadlets can comprise further auxiliary agents (auxiliaries). Depending for what the beadlets are used, the auxiliary agent(s) can vary. These auxiliary agents can be useful for the formulation by further improving its properties, such as physical stability, storage stability, visual perception, etc. Auxiliaries can also be useful for the application in the food or feed product by improving the property of these compositions, physical stability, storage stability, visual perception, controlled release in the GI-tract, pH control, oxidation resistant, etc. The concentration of these auxiliaries can vary, depending on the use of these auxiliaries. These auxiliary agents are usually present in an amount of 0 wt-% to 5 wt-%, based on the total weight of the beadlets.

The beadlets can optionally comprise for example antioxidants.

Antioxidants prevent oxidation of the active ingredients, thus preserving the desired properties of the actives, such as biological activity, color and/or color intensity. Typical antioxidants are vitamin E (tocopherol), vitamin C, ascorbyl palmitate, 2,6-di-tert-butyl-p-cresol (butylated hydroxytoluene or BHT), butylated hydroxyanisole (BHA), ethoxyquin (EMQ), and others. These compounds can be used as such or as mixtures. The beadlets of the present invention comprise 0 to 5 wt-%, based on the total weight of the beadlets, of at least one antioxidant. Therefore a preferred embodiment of the present invention relates to beadlets (XIV), which are beadlets (I), (Ia), (II), (IIa), (III), (IIIa), (IV), (V), (VI), (VII), (VIII), (IX), (X), (XI), (XII), (XII′) and/or (XIII), wherein the beadlets comprise 0 wt-% to 5 wt-%, based on the total weight of the beadlets, of at least one auxiliary agent.

A more preferred embodiment of the present invention relates to beadlets (XIV′), which are beadlets (XIV), wherein the beadlets comprise 0 wt-% to 5 wt-%, based on the total weight of the beadlets, of at least one antioxidant.

An even more preferred embodiment of the present invention relates to beadlets (XIV″), which are beadlets (XIV), wherein the beadlets comprise 0 wt-% to 5 wt-%, based on the total weight of the beadlets, of at least one antioxidant chosen from the group consisting of vitamin E (tocopherol), vitamin C, ascorbyl palmitate, 2,6-di-tert-butyl-p-cresol (butylated hydroxytoluene or BHT), butylated hydroxyanisole (BHA) and ethoxyquin (EMQ).

An especially preferred embodiment of the present invention relates to beadlets (XIV′″), which are beadlets (XIV), wherein the beadlets comprise 0 wt-% to 5 wt-%, based on the total weight of the beadlets, of at least one antioxidant, which vitamin E (tocopherol).

The beadlets are usually produced by using the spray chilled technology or spray cooling technology. This technology is widely known in the field spray drying. It is described for example in trends in Food Science & Technology 15 (2004), 330-347.

The steps of spray chilled or spray cooling technology (process) are:
1) mixing the all ingredients (incl. the wax and/or fats), and
2) atomizing into a chamber, where it is contacted with an air stream which is cool enough to case the droplets to solidify.

It is also preferred that the mixture before atomizing, is grinded. The grinding step can be carried out by various types of mills, i.e. a colloid mill or a ball mill.

The present invention also relates to the production of beadlets (I), (Ia), (II), (IIa), (III), (IIIa), (IV), (V), (VI), (VII), (VIII), (IX), (X), (XI), (XII), (XIII (XIII), (XIV), (XIV′), (XIV″) and/or (XIV′″) by using the spray chilled process or the spray cooling process.

The present invention also relates to the production of beadlets (I), (Ia), (II), (IIa), (III), (IIIa), (IV), (V), (VI), (VII), (VIII), (IX), (X), (XI), (XII), (XII′), (XIII), (XIV), (XIV′), (XIV″) and/or (XIV′″) using the spray chilled process or the spray cooling process, wherein the process comprising the following steps:
1) mixing the all ingredients (incl. the wax and/or fat), and
2) atomizing into a chamber, where it is contacted with an air stream which is cool enough to case the droplets to solidify.

Preferably, the present invention also relates to the production of beadlets (I), (Ia), (II), (IIa), (III), (IIIa), (IV), (V), (VI), (VII), (VIII), (IX), (X), (XI), (XII), (XII′), (XIII), (XIV), (XIV′), (XIV″) and/or (XIV′″) using the spray chilled process or spray cooling process, wherein the process comprising the following steps:
1) mixing the all ingredients (incl. the wax and/or fat), and
2) grinding the mixture of the ingredients (preferably by a colloid mill or a ball mill), and
3) atomizing into a chamber, where it is contacted with an air stream which is cool enough to case the droplets to solidify.

The carotenoid particles (inside the beadlet) do usually have a size (d 0.9) of below 30 μm

When a ball mill is used then the carotenoid particles do usually have a size (d 0.9) of below

The carotene particle size distribution (PSD) inside the beadlets has been determined by laser diffraction”. The PSD was done with analyzer Malvern Mastersizer 2000 and sampler Hydro 2000S. The testing method involve calculation model Fraunhofer with settings “Only Red Laser”, “General Purpose”, “Normal Sensitivity”, “Irregular Shape”, “Obscuration 10-15%”. Before measure the sample a 1 wt % stock solution has to be prepared in hot oil (i.e. corn oil). Oil temperature is 10-15° C. higher than melting point of the wax or fat the beadlet comprise of. If a matrix mixture is used the oil temperature is defined by the wax or fat with the highest melting point. After the coating material is solved and beta carotene is released the suspension is added to Hydro 2000S until the obscuration is reached and remains stable.

Therefore, the present invention also relates to beadlets (XV), which are beadlets (I), (Ia), (II), (IIa), (III), (IIIa), (IV), (V), (VI), (VII), (VIII), (IX), (X), (XI), (XII), (XII′), (XIII), (XIV), (XIV′), (XIV″) and/or (XIV′″), wherein the carotenoid particles (inside the beadlets) have a size (d 0.9) of below 30 μm.

Therefore, the present invention also relates to beadlets (XV′), which are beadlets (I), (Ia), (II), (IIa), (III), (IIIa), (IV), (V), (VI), (VII), (VIII), (IX), (X), (XI), (XII), (XII′), (XIII), (XIV), (XIV′), (XIV″) and/or (XIV′″), wherein the carotenoid particles (inside the beadlets) have a size (d 0.9) of below 15 μm.

The beadlets according to the present invention can be used in many fields of applications. It can be used in food, feed and personal care products. Preferred is the use in food products, very preferred it the use of beadlets as described above in margarines and in beverages (such as soft drinks). The amount, which is used in such products, depends heavily on the product as well as of the color shade which is desired.

The beadlets (I), (Ia), (II), (IIa), (III), (IIIa), (IV), (V), (VI), (VII), (VIII), (IX), (X), (XI), (XII), (XII′), (XIII), (XIV), (XIV′), (XIV″), (XIV″), (XV) and/or (XV′″) are used in the production of food, feed and personal care products; preferably in the production of food products, more preferably in the production of (soft) drinks and margarines.

It is clear that all other commonly known ingredients for producing food, feed and personal care products are also used in the process.

Therefore the present invention also relates to a process of production of food, feed and personal care products, wherein beadlets (I), (Ia), (II), (IIa), (III), (IIIa), (IV), (V), (VI), (VII), (VIII), (IX), (X), (XI), (XII), (XII′), (XIII), (XIV), (XIV′), (XIV″), (XIV′″), (XV) and/or (XV′) are used.

As already stated above the amount of the beadlets (I), (Ia), (II), (IIa), (III), (IIIa), (IV), (V), (VI), (VII), (VIII), (IX), (X), (XI), (XII), (XII′), (XIII), (XIV), (XIV′), (XIV″), (XIV′″), (XV) and/or (XV′) used in the production of food, feed and personal care products depends on the product. Usually the amount of the at least one carotenoid in the food, feed and personal care product is 1 to 12 ppm (=mg carotenoid per kg product).

In a soft drink the amount of the at least one carotenoid is 1 to 12 ppm.

In a margarine the amount of the at least one carotenoid is 1 to 12 ppm.

Therefore the present invention also relates to a process of production of food, feed and personal care products, wherein beadlets (I), (Ia), (II), (IIa), (III), (IIIa), (IV), (V), (VI), (VII), (VIII), (IX), (X), (XI), (XII), (XII′), (XIII), (XIV), (XIV′), (XIV″), (XIV′″), (XV) and/or (XV′) are used and wherein the amount of at least one carotenoid in the food, feed and personal care products is 1 ppm to 12 ppm.

A further embodiment of the present relates to food, feed and personal care products obtained from a process as described above.

As mentioned above the advantages of the beadlets are also that they allow to producing end-market products (consumer products, which are sold i.e. in (grocery) shops), which have good color saturation as well as good color stability (during storage).

The invention is illustrated by the following Examples. All temperatures are given in ° C. and all parts and percentages are related to the weight.

### EXAMPLE 1

161 kg of Revel C (hydrogenated, refined vegetable fat, made out of palm oil, with a melting point of 58 to 62° C.) have been put into a vessel and heated up to 85° C. under inert gas. 1.6 kg dl-α-tocopherol has been added under stirring to the melted “Revel C”. Afterwards 80 kg of crystalline β-carotene was added to the reaction mixture. The mixture is stirred and afterward at 85° C. grinded by using a colloid mill. In a second grinding step the β-carotene was further reduced in particle size by use of an agitated ball mill (Netzsch, LMZ 2), filled with 0.7 mm yttrium stabilized zirconium beads. The final suspension was than sprayed with a temperature of 90° C. The applied main air (bottom air) in the tower had a temperature of 5° C. To stabilize the spraying process a small amount of 85° C. hot air were added from the top of the tower to the spray nozzle. Non-sticky and non-dusty beadlets with a particle size (d 0.1 ) of 267 μm, (d 0.5) of 373 μm, (d 0.9) of 520 μm have been obtained. The particle size of the carotenoid particles (inside the beadlets) was (d 0.1) of 1 μm, (d 0.5) of 3 μm, (d 0.9) of 9 μm.

### EXAMPLE 2

150 kg of Parteck LUB STA 50 (stearic acid 50 vegetable grade with a melting point in the range of 68 to 70° C.) have been put into a vessel and heated up to 85° C. under inert gas. 1.5 kg dl-α-tocopherol has been added under stirring to the melted Parteck LUB STA 50.Afterwards 75 kg of crystalline β-carotene was added to the reaction mixture. The mixture is stirred and afterward at 85° C. grinded by using a colloid mill. In a second grinding step the β-carotene was further reduced in particle size by use of an agitated ball mill (Netzsch, LMZ 2), filled with 0.7 mm yttrium stabilized zirconium beads. The final suspension was than sprayed with a temperature of 90° C. The reaction mixture was spray dried by using the spray chilling technology. The applied main air (bottom air) in the tower had a temperature of 5° C. To stabilize the spraying process a small amount of 85° C. hot air were added from the top of the tower to the spray nozzle. Non-sticky and non-dusty beadlets with a particle size (d 0.1) of 302 μm, (d 0.5) of 414 μm, (d 0.9) of 565 μm have been obtained. The particle size of the carotenoid particles (inside the beadlets) was (d 0.1) of 1 μm, (d 0.5) of 4 μm, (d 0.9) of 10 μm.

### EXAMPLE 3

30.14 kg of Revel A (fractionated, not hydrogenated, refined vegetable fat, made out of palm oil, with a melting point of 58 to 62° C.) have been put into a vessel and heated up to 85° C. under inert gas. 0.32 kg dl-α-tocopherol has been added under stirring to the melted Revel A. Afterwards 15 kg of crystalline β-carotene was added to the reaction mixture. The mixture is stirred and afterward at 85° C. grinded by using a colloid mill. The suspension was than sprayed with a temperature of 90° C. The reaction mixture was spray dried by using the spray chilling technology. The applied main air (bottom air) in the tower had a temperature of 5° C. To stabilize the spraying process a small amount of 85° C. hot air were added from the top of the tower to the spray nozzle. Non-sticky and non-dusty beadlets with a particle size (d 0.1) of 263 μm, (d 0.5) of 361 μm, (d 0.9) of 495 μm have been obtained. The particle size of the carotenoid particles (inside the beadlets) was (d 0.1) of 2 μm, (d 0.5) of 6 μm, (d 0.9) of 22 μm.

### EXAMPLE 4

25.1 kg of Remi A-212 (fractionated, not hydrogenated vegetable fat, made out of palm oil, with a melting point of 61 to 63° C.) have been put into a vessel and heated up to 85° C. under inert gas. 0.3 kg dl-α-tocopherol has been added under stirring to the melted hydrogenated soybean oil. Afterwards 12.5 kg of crystalline β-carotene was added to the reaction mixture. The mixture is stirred and afterward at 85° C. grinded by using a colloid mill. The suspension was than sprayed with a temperature of 90° C. The reaction mixture was spray dried by using the spray chilling technology. The applied main air (bottom air) in the tower had a temperature of 5° C. To stabilize the spraying process a small amount of 85° C. hot air were added from the top of the tower to the spray nozzle. Non-sticky and non-dusty beadlets with a particle size (d 0.1) of 333 μm, (d 0.5) of 480 μm, (d 0.9) of 688 μm have been obtained. The particle size of the carotenoid particles (inside the beadlets) was (d 0.1) of 2 μm, (d 0.5) of 8 μm, (d 0.9) of 29 μm.

### EXAMPLE 5

30 kg of Sojaöl Hydriert Ph. Eur. 7.0 (hydrogenated soybean oil with a melting point in the range of 68 to 72° C.) have been put into a vessel and heated up to 85° C. under inert gas. 0.46 kg dl-α-tocopherol has been added under stirring to the melted hydrogenated soybean oil. Afterwards 15 kg of crystalline β-carotene was added to the reaction mixture. The mixture is stirred and afterward at 85° C. grinded by using a colloid mill. The suspension was than sprayed with a temperature of 90° C. The reaction mixture was spray dried by using the spray chilling technology. The applied main air (bottom air) in the tower had a temperature of 5° C. To stabilize the spraying process a small amount of 85° C. hot air were added from the top of the tower to the spray nozzle. Non-sticky and non-dusty beadlets with a particle size (d 0.1) of 353 μm, (d 0.5) of 562 82 m, (d 0.9) of 883 μm have been obtained. The particle size of the carotenoid particles (inside the beadlets) was (d 0.1) of 1 μm, (d 0.5) of 4 μm, (d 0.9) of 23 μm.

### EXAMPLE 6

12.55 kg of Remi A-212 (fractionated, not hydrogenated vegetable fat, made out of palm oil, with a melting point of 61 to 63° C.) and 12.55 kg of Remi ST-296 (fractionated, not hydrogenated vegetable fat, made out of palm oil, with a melting point of 52 to 56° C.) have been put into a vessel and heated up to 85° C. under inert gas. 0.3 kg dl-α-tocopherol has been added under stirring to the melt. Afterwards 12.5 kg of crystalline β-carotene was added to the reaction mixture. The mixture is stirred and afterward at 85° C. grinded by using a colloid mill. The suspension was than sprayed with a temperature of 90° C. The reaction mixture was spray dried by using the spray chilling technology. The applied main air (bottom air) in the tower had a temperature of 5° C. To stabilize the spraying process a small amount of 90° C. hot air were added from the top of the tower to the spray nozzle. Non-sticky and non-dusty beadlets with a particle size (d 0.1) of 301 μm, (d 0.5) of 414 μm, (d 0.9) of 567 μm have been obtained. The particle size of the carotenoid particles (inside the beadlets) was (d 0.1) of 2 μm, (d 0.5) of 8 μm, (d 0.9) of 28 μm.

### EXAMPLE 7

30 kg of Rüböl H70 (hydrogenated rapeseed oil with a melting point between 70 to 74° C.) have been put into a vessel and heated up to 90° C. under inert gas. 0.46 kg dl-α-tocopherol has been added under stirring to the melted Rüböl H70 . Afterwards 15 kg of crystalline β-carotene was added to the reaction mixture. The mixture is stirred and afterward at 90° C. grinded by using a colloid mill. The suspension was than sprayed with a temperature of 90° C. The reaction mixture was spray dried by using the spray chilling technology. The applied main air (bottom air) in the tower had a temperature of 5° C. To stabilize the spraying process a small amount of 90° C. hot air were added from the top of the tower to the spray nozzle. Non-sticky and non-dusty beadlets with a particle size (d 0.1) of 387 μm, (d 0.5) of 607 μm, (d 0.9) of 954 μm have been obtained. The particle size of the carotenoid particles (inside the beadlets) was (d 0.1) of 1 μm, (d 0.5) of 4 μm, (d 0.9) of 21 μm.

The beadlets of Example 1-7 can be incorporated into food, feed and personal care product (usually in such an amount that the carotenoid content in such products is 1 to 12 ppm). Preferably they are used in soft drinks or margarines.

1. A process for producing beadlets using a spray chilled process or a spray cooling process, wherein the process comprises the following steps:
(a) forming a mixture by mixing (i) 25 wt-% to 45 wt-%, based on the total weight of the beadlets, of at least one carotenoid and (ii) 55 wt-% to 75 wt-% of a matrix material which comprises at least one unsaturated (non-hydrogenated), partially saturated (partially hydrogenated) or fully saturated (fully hydrogenated) wax and/or fat having a melting point of from 40° C. to 85° C., and
(b) atomizing the mixture of the at least one carotenoid and the matrix material into a chamber and contacting the atomized mixture within the chamber with an air stream which is sufficiently cool to cause droplets of the mixture to solidify and thereby form beadlets having an average particle diameter of 50 μm to 1000 μm and with the at least one carotenoid having a particle size (d0.9) inside the beadlets of below 30 μm.

2. The process according to claim 1, which further comprises grinding the mixture of the at least one carotenoid and matrix material before atomizing.

3. The process according to claim 1, wherein the with the proviso that, beadlets comprising:
(i) 33 wt-% to 34 wt-%, based on the total weight of the beadlets, of β-carotene, and
(ii) 66 wt-% to 67 wt-%, based on the total weight of the beadlets, of glycerin monostearate,
are excluded.

4. The process according to claim 1, wherein the at least one carotenoid is present in the mixture in an amount of 25-40 wt-%, based on the total weight of the beadlets=.

5. The process according to claim 1, wherein the at least one carotenoid is present in the mixture in an amount of 25-35 wt-%, based on the total weight of the beadlets.

6. The process according to claim 1, wherein the melting point of the matrix material is 45° C. to 80° C.

7. The process according to claim 1, wherein the carotenoid is at least one selected from the group consisting of α-carotene, β-carotene, 8′-apo-β-carotenal, 8′-apo-β-carotenoic acid esters, canthaxanthin, astaxanthin, lycopene, lutein, zeaxanthin and crocetin.

8. The process according to claim 1, wherein the at least one carotenoid is β-carotene.

9. The process according to claim 1, wherein the wax and/or fat is at least one selected from the group consisting of glycerin monostearate, candelilla wax, palmitic acid, stearic acid, glycerintristearate, glycerin monobehenate, glycerin dibehenate, glycerin tribehenate, behenic acid, polyclyceryl palmito stearate, hydrogenated and nonhydrogenated refined vegetable fat made from palm oil, and hydrogenated rapeseed oil.

10. The process according to claim 1, wherein step (a) comprises mixing at least one further auxiliary agent with the at least one carotenoid and the matrix material.

11. The process according to claim 10, wherein the auxiliary agent comprises at least one antioxidant.

12. The process according to claim 11, wherein the antioxidant is at least one selected from the group consisting of vitamin E, vitamin C, ascorbyl palmitate, 2,6-di-tert-butyl-p-cresol, butylated hydroxyanisole and ethoxyquin.

13. The process according to claim 10, wherein the at least one further auxiliary agent is present in an amount of 0 wt-% to 5 wt-%, based on the total weight of the beadlets.

14. A process for the production of food, feed and personal care products, wherein the process comprises incorporating into a food, feed or personal care product an effective amount of beadlets comprising:
(i) 55 wt-% to 75 wt-%, based on the total weight of the beadlets, of a matrix material, and
(ii) 25 wt-% to 45 wt-%, based on the total weight of the beadlets, of carotenoid particles comprised of at least one carotenoid within the matrix material, wherein
the matrix material comprises at least one unsaturated (non-hydrogenated), partially saturated (partially hydrogenated) or fully saturated (fully hydrogenated) wax and/or fat having a melting point of from 40° C. to 85° C., and wherein
the beadlets have an average particle diameter of 50 μm to 1000 μm, and wherein
the carotenoid particles within the matrix material of the beadlets have a particle size (d0.9) of below 30 μ.

15. The process according to claim 14, wherein the amount of the at least one carotenoid in the food, feed and personal care product is 1 to 12 ppm.

16. The process according to claim 14, wherein a food product is produced.

17. The process according to claim 16, wherein the food product is a soft drink.

18. The process according to claim 16, wherein the food product is a margarine.
